(19) United States
(12) Patent Application Publication
(10) Pub. No.: US 2024/0198080 A1
(43) Pub. Date: Jun. 20, 2024

(54) IMPROVED CENTRIFUGAL BLOOD PUMP

(71) Applicants: Zhongjun WU, Marriottsville, MD (US); Bartley P. GRIFFITH, Gibson Island, MD (US); Jiafeng ZHANG, Silver Spring, MD (US); Charles Luddy, Alexandria, VA (US)

(72) Inventors: Zhongjun WU, Marriottsville, MD (US); Bartley P. GRIFFITH, Gibson Island, MD (US); Jiafeng ZHANG, Silver Spring, MD (US); Charles Luddy, Alexandria, VA (US)

(21) Appl. No.: 18/554,585

(22) PCT Filed: Apr. 11, 2022
(86) PCT No.: PCT/US22/24233

§ 371 Date: Oct. 9, 2023

Related U.S. Application Data
(60) Provisional application No. 63/172,882, filed on Apr. 9, 2021.

(51) Int. Cl. A61M 60/232; A61M 60/109; A61M 60/419; A61M 60/422; A61M 60/538; A61M 60/825

(52) U.S. Cl. CPC A61M 60/232; A61M 60/109; A61M 60/419; A61M 60/422; A61M 60/538; A61M 60/825

(57) ABSTRACT

A blood flow pump device includes a centrifugal pump having a single ball-and-cup hybrid magnetic/blood immersed bearing-supported shrouded impeller driven by a magnetically coupled motor drive. The device further includes a housing, an impeller, and a shroud, thus forming a pump chamber. The impeller is a shrouded impeller having extended blades from a hub of the impeller and the shroud. The system and method employ a blood flow pump device having a housing with a blood inlet and a blood outlet, the housing defining a fluid pathway between the inlet and the outlet, and the housing containing an impeller comprising a base, a base lid, two blades, and a shroud, with the housing positioned on a motor. A controller communicates with the motor and controls rotational speed of the impeller in response to the blood flow through the blood outlet.

### STATEMENT OF GOVERNMENT INTEREST

This invention was made with government support under grant numbers HL118372 and HL141817 awarded by the National Institutes of Health. The government has certain rights in the invention.

### FIELD OF THE INVENTION

The current invention relates to blood flow pump devices and systems and to methods of their use, and more particularly to blood flow pump devices that have reduced potential of blood damage compared to typical blood flow pump devices.

### BACKGROUND OF THE INVENTION

Blood pumps are commonly used in mechanically assisted circulation for ventricular assistance for cardiac failure or for respiratory or cardiopulmonary ECMO support or during cardiopulmonary bypass (CPB) for cardiac surgery. Over the past several decades, a wide variety of mechanical blood pumps have been invented and evolved, including roller pumps, pulsatile displacement pumps, centrifugal flow pumps and axial flow pumps. Centrifugal pumps appear to have almost completely substituted roller pumps for CPB and ECMO applications because of their advantage of decreased trauma to red blood cells and a less pronounced systemic inflammatory response compared with roller pumps. Currently, the CentriMag pump (Abbott, Chicago, IL, USA) and Rotaflow pump (Getinge, Gothenburg, Sweden) are the two clinical centrifugal blood pumps commonly used in extracorporeal circulatory support or ECMO support. The CentriMag blood pump employs a bearingless impeller technology and it does not contain seals or bearings that are considered to be the potential cause of the thrombus formation. The Rotaflow pump is a shrouded impeller pump that employs a magnetically stabilized impeller on a monopivot and features a peg-top, one-point, sapphire bearing which were considered to lower friction.

The high-speed rotation of the impeller in a centrifugal pump inevitably creates regions of non-physiological shear stress (NPSS) within the pump. The NPSS can cause damage to blood cells, leading to altered blood function contributing to hemolysis, thrombosis and bleeding complications. In the past, computational fluid dynamics (CFD) and experimental studies have been proved to be an efficient way to investigate the flow feature of blood pumps and also to guide the pump design and optimization. Thus, there is an unmet need for pumps that operate with reduced damage to blood cells and lower likelihood of thrombosis.

### SUMMARY OF THE INVENTION

Provided according to certain aspects of an embodiment is a blood flow pump device configured to have improved flow features and reduced potential of blood damage, compared to typical devices. In certain configurations, the device was tested using computational, experimental, and combined methods to investigate the flow features and blood damage potentials compared to typical pumps. For example, flow features include blood flow structure, shear stress levels, flow washout, hemolysis index, and the like. As a further example, a blood flow pump configured in accordance with aspects of the invention was tested for hemodynamic and hemolytic performance under an operating condition relevant to ECMO support (flow: 5 L/min, pressure head: ˜350 mmHg). Furthermore, a blood flow pump configured in accordance with aspects of the invention may have a smaller area-averaged wall shear stress (WSS), a smaller volume with a scalar shear stress (SSS) level greater than 100 Pa and a lower device-generated hemolysis index compared to typical pumps. A blood flow pump configured in accordance with aspects of the invention may also have better calculated residence times and washout than typical pumps. Still further, experimental data from in-vitro hemolysis testing suggests that a blood flow pump configured in accordance with aspects of the invention may have more preferable normalized hemolysis index (NIH) than typical pumps.

According to aspects of an exemplary embodiment, the blood flow pump device is configured to mechanically assist circulation for ventricular assistance and extracorporeal membrane oxygenation support or during cardiopulmonary bypass for cardiac surgery. The blood flow pump may be configured to reduce bleeding and thrombosis complications in patients associated with typical blood pumps. The pump may comprise an extracorporeal centrifugal blood pump having a hybrid magnetic and mechanical bearing configured to reduce device-induced blood trauma. The bearing may comprise a sapphire ball and ultrahigh molecular weight polyurethane cup configured to reduce rotational friction and material abrasion. The bearing may be configured to have a conical-like cross-section, thus forming a smooth transition between the cup bearing and the bearing, to reduce the likelihood of stagnant flow.

Still other aspects, features and advantages of the invention are readily apparent from the following detailed description, simply by illustrating a number of particular embodiments and implementations, including the best mode contemplated for carrying out the invention. The invention is also capable of other and different embodiments, and its several details can be modified in various obvious respects, all without departing from the spirit and scope of the invention. Accordingly, the drawings and description are to be regarded as illustrative in nature, and not as restrictive.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized. The present invention is illustrated by way of example, and not by way of limitation, in the figures of the accompanying drawings, in which like reference numerals refer to similar elements, and in which:

FIG. 1 is an illustration of a centrifugal pump according to certain aspects of an embodiment of the invention.

FIG. 2 is an illustration of one typical pump known in the art.

FIG. 3 is an illustration of another typical pump known in the art.

FIG. 4 contains three charts of pressure head (AP) as measured and CFD predicted of the following running at different blood flow rates and rotational speeds: (a) an embodiment of the device illustrated in FIG. 1, (b) a typical pump illustrated in FIG. 2, and (c) a typical pump illustrated in FIG. 3;

FIG. 5 is an illustration of streamlines of relative velocity fields for a) one embodiment of the device illustrated in FIG. 1, and b) and c) typical pumps running under a pressure head of 350 mmHg and flow rate of 5 L/min;

FIG. 6a is a drawing illustrating wall shear stress (WSS) of one embodiment of the device in FIG. 1 (left) and the typical pumps in FIGS. 2 and 3 (middle and right) running under a pressure head of 350 mmHg and flow rate of 5 L/min;

FIG. 6b is a drawing illustrating scalar shear stress (SSS) distributions of one embodiment of the device in FIG. 1 (left) and the typical pumps in FIGS. 2 and 3 (middle and right) running under a pressure head of 350 mmHg and flow rate of 5 L/min;

FIG. 7a is a drawing illustrating volumes with different SSS levels of one embodiment of the device in FIG. 1 and typical pumps in FIGS. 2 and 3 running under a pressure head of 350 mmHg and flow rate of 5 L/min;

FIG. 7b is a drawing illustrating averaged residence times of one embodiment of the device in FIG. 1 and typical pumps in FIGS. 2 and 3 running under a pressure head of 350 mmHg and flow rate of 5 L/min;

FIG. 8 is an illustration of simulated hemolysis index (HI) contours of one embodiment of the device in FIG. 1 (left) and typical pumps in FIGS. 2 and 3 (middle and right) running under a pressure head of 350 mmHg and flow rate of 5 L/min;

FIG. 9a is a chart of CFD-predicted HI levels at the exit of one embodiment of the device in FIG. 1 and the typical pumps in FIGS. 2 and 3 operated to under a pressure head of 350 mmHg and flow rate of 5 L/min;

FIG. 9b is an illustration of experimentally measured NIH of one embodiment of the device in FIG. 1 and the typical pumps in FIGS. 2 and 3 operated to under a pressure head of 350 mmHg and flow rate of 5 L/min;

FIG. 10 is a cross-sectional view of one embodiment of the device illustrated in FIG. 1;

FIG. 11a is a perspective view of one embodiment of the inlet 100 and the pump housing 220 of the current device illustrated in FIG. 1;

FIG. 11b is a cross-sectional view of a pump housing 220 illustrated in FIG. 11a;

FIG. 12a is a perspective view of a shrouded impeller of the pump shown in FIG. 10;

FIG. 12b is a cross-sectional view of the shrouded impeller shown in FIG. 12a;

FIG. 13 is a perspective view of one embodiment of a housing 220 shown in FIG. 11a;

FIG. 14 is a bottom view of the blade profile of the shrouded impeller shown in FIG. 12a without a base 273;

FIG. 15 is a top view of the blade profile of the shrouded impeller shown in FIG. 12a without the shroud 271;

FIG. 16 is a perspective view of the blade profile of the shrouded impeller shown in FIG. 15;

FIG. 17 is a top view of the base lid 273a shown in FIG. 16;

FIG. 18 is a perspective view of the base lid 273a shown in FIG. 17;

FIG. 19 is a perspective view of the base 273 shown in FIG. 16;

FIG. 20 is an isometric view of the assembly of the base 273 and the base lid 273a;

FIG. 21 is an isometric view of the assembly of the half-ball bearing 400 and the cup bearing 500 shown in FIG. 10;

FIG. 22 is a cross-sectional view of the pump shown in FIG. 10 attached to an external motor;

FIG. 23a is an illustration of a cross-sectional view of a blood flow field within the pump of FIG. 10; and

FIG. 23b is an illustration of a cross-sectional view of a blood flow field within a bearing area of the pump of FIG. 10.

### DETAILED DESCRIPTION

The following detailed description is provided to gain a comprehensive understanding of the methods, apparatuses and/or systems described herein. Various changes, modifications, and equivalents of the systems, apparatuses and/or methods described herein will suggest themselves to those of ordinary skill in the art.

Descriptions of well-known functions and structures are omitted to enhance clarity and conciseness. The terminology used herein is for the purpose of describing particular embodiments only and is not intended to be limiting of the present disclosure. As used herein, the singular forms “a”, “an” and “the” are intended to include the plural forms as well, unless the context clearly indicates otherwise. Furthermore, the use of the terms a, an, etc. does not denote a limitation of quantity, but rather denotes the presence of at least one of the referenced items.

The use of the terms “first”, “second”, and the like does not imply any particular order, but they are included to identify individual elements. Moreover, the use of the terms first, second, etc. does not denote any order of importance, but rather the terms first, second, etc. are used to distinguish one element from another. It will be further understood that the terms “comprises” and/or “comprising”, or “includes” and/or “including” when used in this specification, specify the presence of stated features, regions, integers, steps, operations, elements, and/or components, but do not preclude the presence or addition of one or more other features, regions, integers, steps, operations, elements, components, and/or groups thereof.

Although some features may be described with respect to individual exemplary embodiments, aspects need not be limited thereto such that features from one or more exemplary embodiments may be combinable with other features from one or more exemplary embodiments.

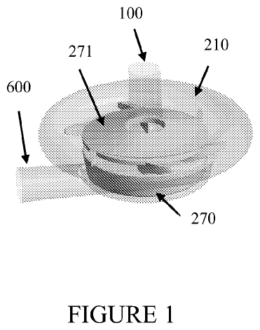

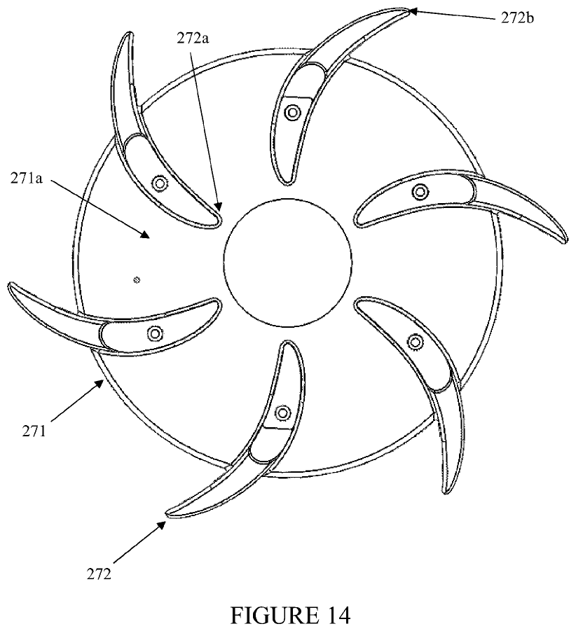

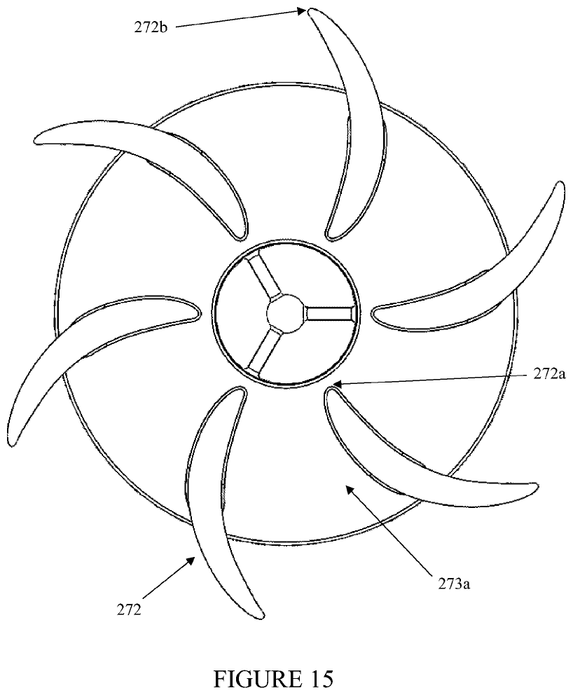

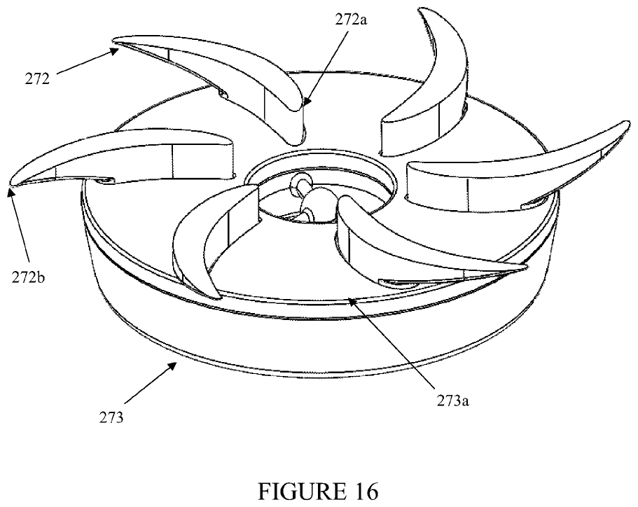

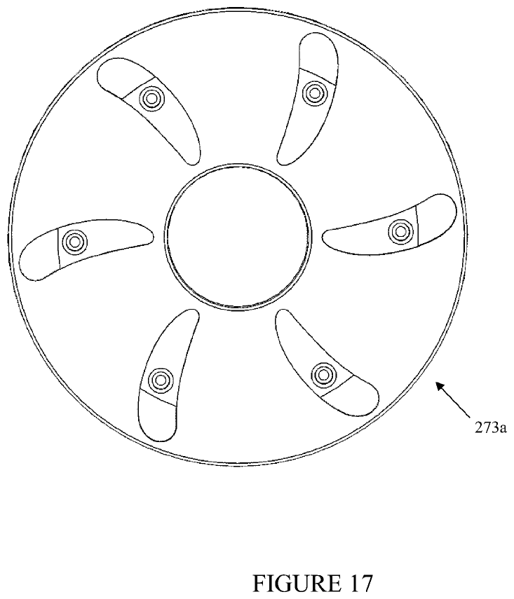

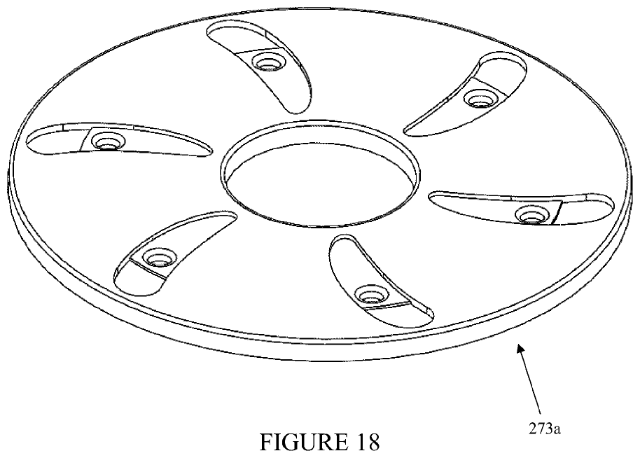

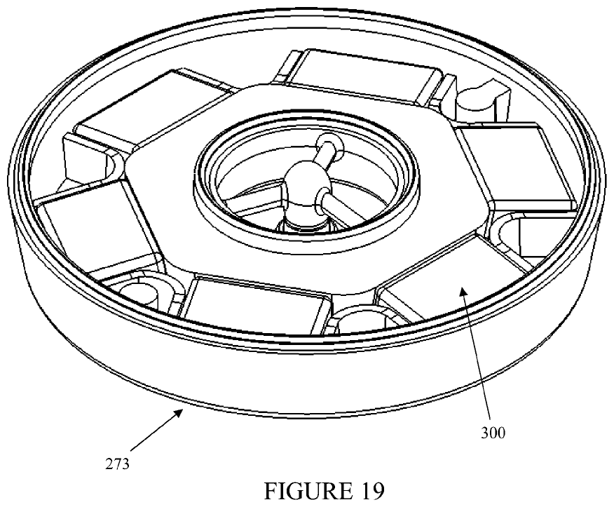

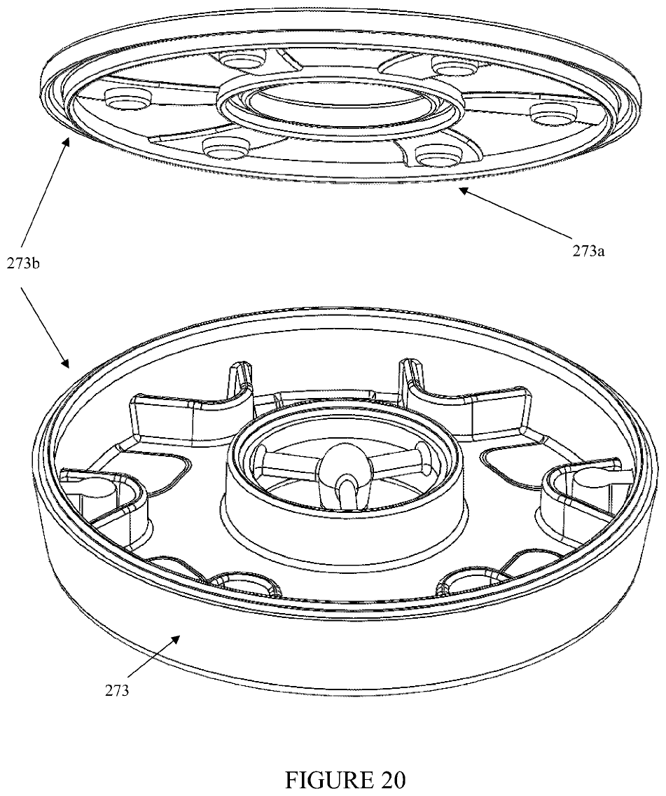

Provided according to certain aspects of an embodiment of the invention is a blood pump device. Referring to the figures, including FIGS. 1 and 10-23, the device includes a centrifugal pump having a single ball-and-cup hybrid magnetic/blood immersed bearing-supported shrouded impeller driven by a magnetically coupled motor drive. The device further includes a housing, an impeller, and a motor drive, thus forming a functioning pump. The impeller is a shrouded impeller having blades extending from a hub of the impeller and the shroud. The primary flow path extends from an inlet of the device to an outlet of the device through the impeller blade passage between the hub and the shroud of the impeller. The outlet is generally tangentially-oriented with respect to the primary flow path. In another embodiment, the device includes a secondary flow path generally forming a U-like shape. The secondary flow path is generally formed in a gap between the rotating impeller hub and the stationary housing. In still another embodiment, the secondary flow path merges with the primary flow path through a central opening in the impeller.

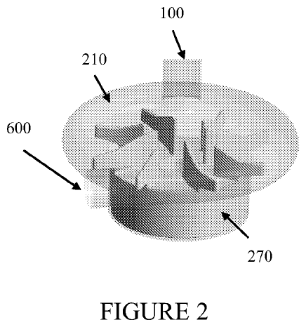

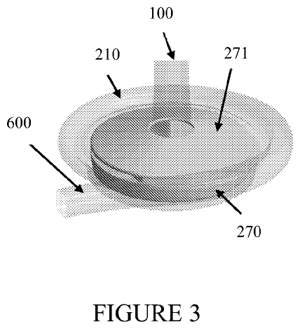

Still referring to FIGS. 1 and 10-23, the shroud 271 and the impeller 270 are configured to reduce net axial forces on the shroud 271 and the impeller 270, compared to typical pumps, such as those in FIGS. 2 and 3. For example, each of the shroud 271 and a base 273 of the impeller 270 have a substantially equally inner diameter and outer diameter such that an axial lifting force on the shroud 271 and the impeller base 273 is minimized. As a further example, the impeller 270 can have an outer diameter between 5 mm and 100 mm, depending on operating requirements of the pump. Furthermore, the impeller 270, housing 220, and shroud 271 can be formed by injection molding using typical medical-grade materials, such as polycarbonate or the like.

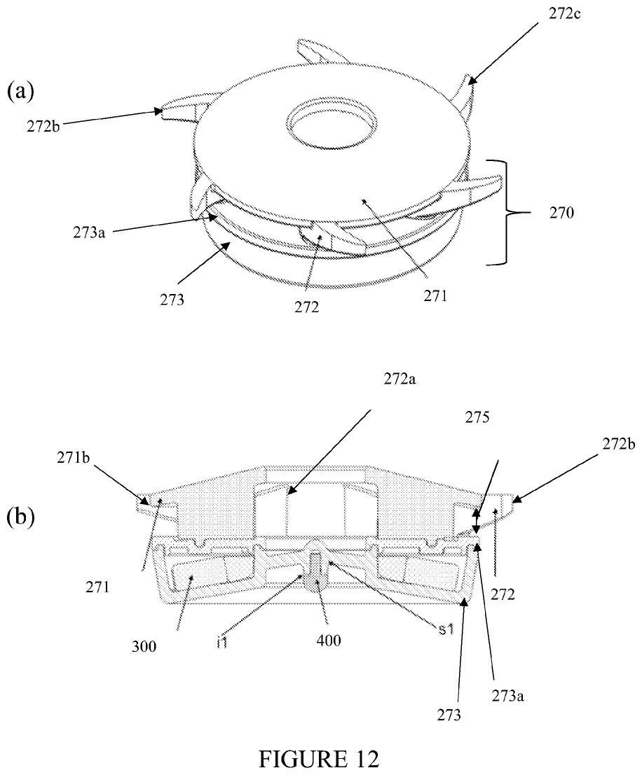

Now referring to FIGS. 12a and 12b, and according to one embodiment of the device, the impeller 270 includes a set of blades 272 and the annular base 273. The impeller blades 272 are configured to minimize shear stress applied on blood flowing through the pump. In one embodiment, the impeller 270 has from two to eight blades 272 and are evenly distributed on a top portion of the annular base 273. The blades 272 further exhibit a streamlined profile, such as determined by computational fluid dynamics simulations, to reduce shear stress applied on blood flowing through the device. In one example, each of the blades 272 have leading edges 272a that are oriented at approximately 90 degrees with respect to a circumferential direction. In another example, each of the blades have trailing edges 272b that are oriented at approximately 40 degrees with respect to a circumferential direction. In another embodiment, each of the blades 272 extend beyond an outer diameter of the impeller base 273 and the shroud 271. Thus, the impeller blades 272 are configured to minimize shear stress applied on blood flowing through the pump.

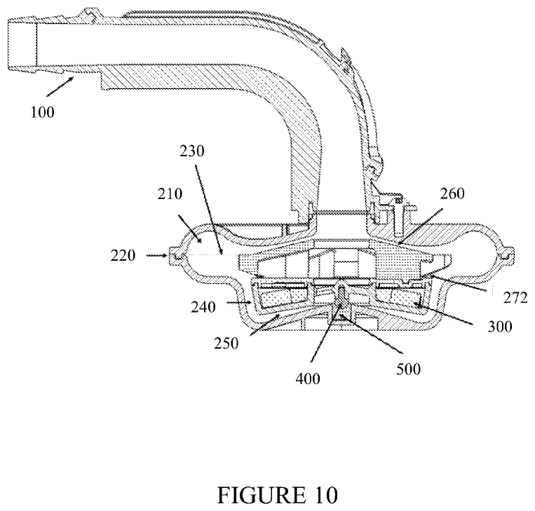

Still referring to FIGS. 12a and 12b, in one example of the impeller 270, one or more of the blades 272 have variable thickness, such as a thickness that is smaller at a leading edge 272a and a trailing edge 272b compared to a middle portion. As a further example, each of the blades have a thickness at the middle portions (e.g., about 40%-50% of a length of each blade) that is approximately 1.5 times the thickness at the leading edge 272a of each blade. In a still further example, the thickness of the blades varies generally according to a smooth curvilinear surface between the leading edge 272a and the middle portion, and between the middle portion and the trailing edge 272b (e.g., a smooth curvature from the leading edge to the trailing edge). In certain configurations, each of the leading edges 272a of the blades 272 has a height that is from 3 mm to 9 mm, and more preferably approximately 7 mm. In another embodiment, each of the trailing edges 272b of the blades 272 has a height that is from 1 mm to 5 mm, and more preferably approximately 2.5 mm. In certain configurations, each of the blades 272 has a height that generally continuously reduces from approximately 7 mm at a tip of each blade's leading edge 272a to approximately 3.5 mm at the outer edge of the impeller. Likewise, in certain configurations, a portion of each of the blades 272 that extend beyond the outer diameter of the annular base 273 have a top surface 272c with a generally flattened profile (compared to portions of each blade within the outer diameter of the annular base). Furthermore, and as described herein, the top surface 272c of each blades' trailing edge 272b is generally tangential to the center plane 230 of the volute 210 (as best viewed in FIG. 10).

Now referring to FIGS. 10 and 12, the impeller 270 includes a shroud 271 that is configured to balance hydrodynamic forces caused by a pressure difference (e.g., pressure gradient from a bottom of the impeller base 273 to the blades 272) that tends to lift the impeller 270. For example, the secondary flow path (i.e. along a bottom surface of the impeller hub) has a pressure that is larger than the pressure in blade channels. This pressure difference can generate an axial lifting force on the impeller. The shroud 271 reduces the net pressure difference between the top surface of the shroud 271 and the impeller 270 bottom surface to reduce axial lifting force of the impeller 270 such that the impeller 270 experiences minimal lift. In one embodiment, the top of the shroud 271 slopes downward from approximately the inner diameter of the shroud 271 to the outer diameter of the shroud 271. In one embodiment, the slope of the shroud 271 is from 0° to 30° downward, and more preferably approximately 14°, as measured from a plane parallel to the inner diameter of the shroud 271. In certain configurations, the bottom of the impeller base 273 slopes downward from approximately the inner diameter of the base 273 to the bottom lower edge of the base 273. In certain configurations, the slope of the bottom of the base 273 is from 5° to 15° downward, and more preferably approximately 10°, as measured from a plane parallel to the inner diameter of the base 273.

Furthermore, the impeller 270 is configured to reduce shear stress levels, such as by having a variable gap size between the housing 220 and the impeller 270. For example, where the impeller circumferential speed is higher (e.g., radially distal from a central axis of the impeller), a distal gap g1 240 may have a size that is larger than where the impeller circumferential speed is lower, such as at intermediate gap g2 250 or at a radially proximal gap g3 260.

In an exemplary embodiment, the intermediate gap g2 250 (e.g., between the impeller bottom surface and the top of the bottom housing) has a width that is generally reduced according to reducing radial distance from the central axis of the impeller 270. Thus, shear stress of the blood at a greater radial distance from the central axis of the impeller 270 is reduced at a given radii, compared to typical pumps.

In an exemplary embodiment, the proximal gap g3 260 (i.e. between the shroud and the upper housing) has a width that is configured to prevent the impeller 270 from being dislodged from a cup bearing 500 (at a central bottom portion of the housing 220). In certain configurations, the proximal gap g3 260 also gradually reduces generally proportionally to the radial distance from the central axis of the impeller 270. For example, referring to FIG. 10, the gap size at a proximal location 260 near the central hole of the impeller is approximately 0.75 mm.

Still referring to FIGS. 10 and 12a and 12b, in certain configurations, distal gap g1 240 has a width that is approximately 2 mm. Further in certain configurations, the intermediate gap g2 250 width is approximately between 0.5 mm and 2 mm. Still further in certain configurations, the proximal gap g3 260 width is approximately between 2 mm and 0.75 mm.

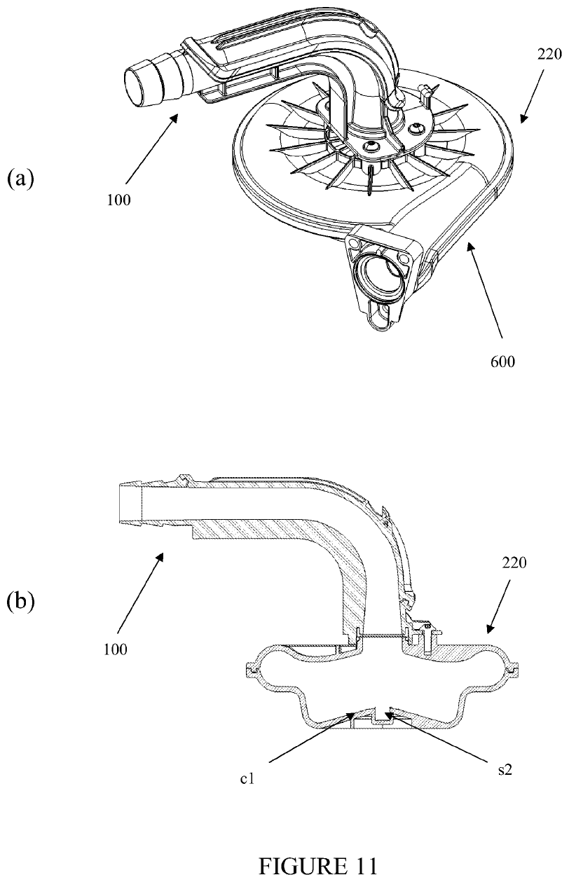

Now referring to FIGS. 10 and 11 and in accordance with further aspects of an exemplary embodiment, the pump housing 220 has a concave shape extending from the inside of the pump housing outward to form a volute 210. The center of the volute 210 to the interior surface of the pump housing may have a radius of 2 to 6 mm, and more preferably 5 mm.

Now referring to FIGS. 10-20, 22 and 23, an exemplary embodiment of the impeller comprises a channel in approximately the center of the impeller 270, and the axis of the channel is approximately perpendicular to the center plane of volute 230. In certain configurations, the blades 272 are situated between the bottom of the shroud 271 and the base lid 273a in such a manner that there is a shroud-impeller (interior) gap 275 at the outer diameter of 1.5 to 6 mm between the bottom of the shroud 271 and the top of the base lid 273a, such as preferably 3.5 mm.

Now referring to FIGS. 10, 11b, 12a, 12b, 21, 22, and 23b, the pump according to aspects of an exemplary embodiment includes a bearing. The bearing includes a cup 500 and a ball 400, in which the ball 400 is configured to fit and rotate at a bearing interface at a first end of the cup 500. In one embodiment, the cup 500 and ball 400 are configured to reduce thrombosis by being shaped to increase washing by blood flow (e.g., by the vertical primary flow, described below). The cup 500 is generally conical and has a cup-like (e.g., concave) profile shape at the first end. The cup 500 is positioned such that the first end between the ball 400 and the cup 500 is washed completely by a vertical primary flow (see FIG. 10 and further described below).

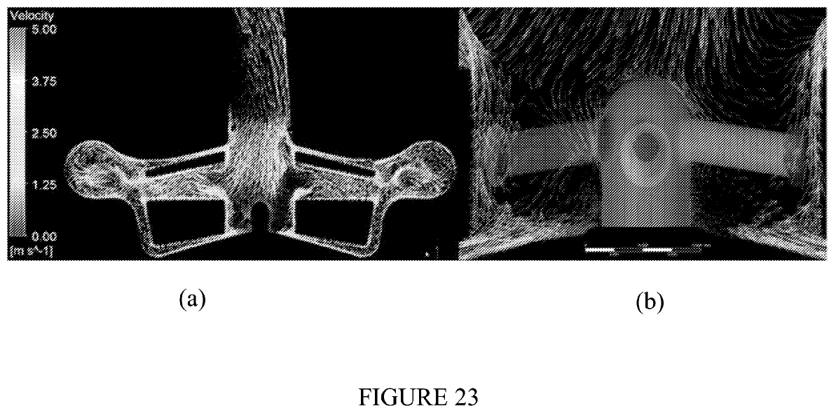

Still referring to FIGS. 10, 11b, 12a, 12b, 21, 22, and 23b, the cup 500 is press-fit into a center opening s2 (i.e. along the central axis of the housing 220 that is perpendicular to the center plane of volute 230) of a bottom portion of the housing 220 (see FIG. 11b) such that the first end of the cup 500 is elevated above the bottom portion of the housing 220 (see FIG. 10). As further described herein, the first end of the cup 500 is positioned above a bottom surface cl of the housing 220. As further described below and shown in FIG. 11b, the bottom surface cl of the housing 220 has a conical profile configured to reduce direct (e.g., head-to-head) interaction between the primary flow and the secondary flow. The bottom surface cl of the housing 220 is further configured to position the bearing interface above the bottom surface cl of the housing such that it is washed completely by the vertical primary flow (see FIG. 23b). Thus, the pump is configured to have a primary blood flow with sufficiently large velocity at the bearing interface to substantially reduce heat generated by friction between the rotating ball 400 and cup 500 and reduce locally high temperature that can cause hemolysis and thrombosis.

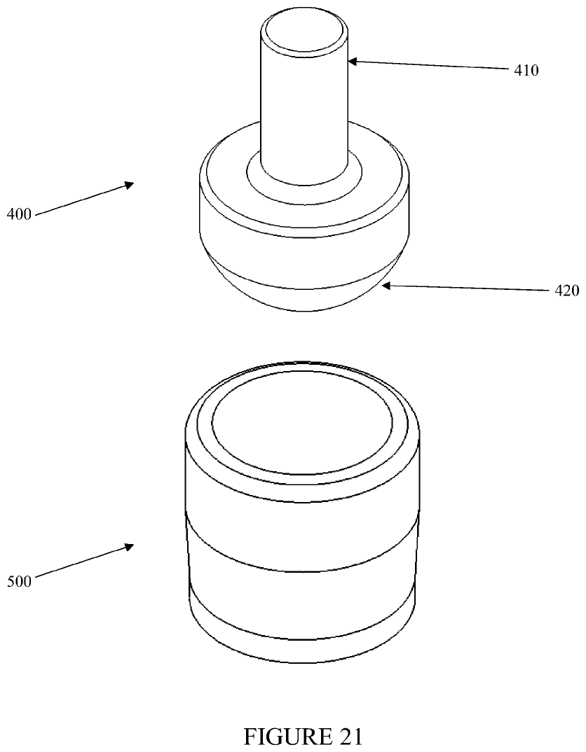

Referring again to FIGS. 12b and 21, in certain configurations, the ball 400 is formed of typical biocompatible bearing materials, such as sapphire. Furthermore, the ball 400 has an upper portion 410 and a tip 420. The upper portion 410 is configured to couple (e.g., press-fit) to the impeller 270 at i1 forming a smooth surface between the ball 400 and the impeller 270. The upper portion 410 has a generally cylindrical body. The tip 420 has a convex, half-ball like shape and diameter that is approximately similar to a diameter of the cup 500 to smoothly rotatably engage the cup 500. The ball 400 and cup 500 rotatably engage at the bearing interface with reduced friction compared to typical pumps. The ball 400 and cup 500 further engage to form a smooth surface that reduces the likelihood of stagnant blood flow. Thus, the bearing is configured to reduce heat damage and thrombosis in the blood flow compared to typical pumps.

Still referring to FIGS. 12b and 21, the cup 500 and ball 400 may be formed of typical biocompatible bearing materials. In certain configurations the cup 500 is formed of ultra-high molecular weight polyethylene (UHMWPE). The cylindrical body of the ball 400, such as a sapphire ball, can be press fit into the impeller 270 supporting structure at s1 without adhesives or fasteners, for example.

Referring to FIGS. 10, 11b, 12b, 13, and 22, the housing is configured to circumferentially magnetically couple the impeller 270 to the external motor drive 700. The housing bottom cl and the bottom of the impeller base 273 have a conical-like shape through which the impeller base 273 and external motor drive 700 couple. The conical-like shape and the magnetic coupling substantially reduce a net axial force between the housing 220 and impeller 270, compared to typical pumps. Thus, the housing 220 and impeller 270 are configured to reduce friction force at the bearing interface to reduce blood-damaging heat, compared to typical pumps.

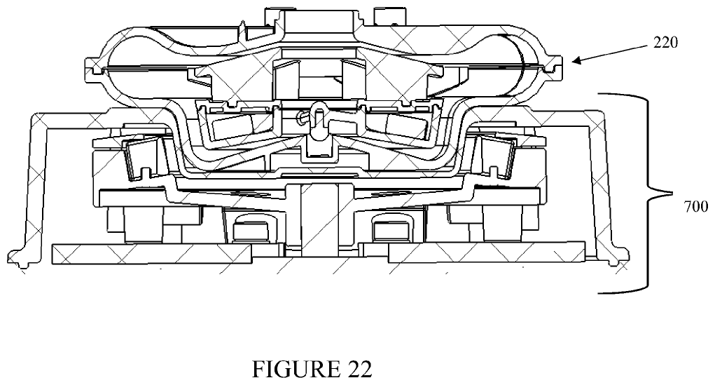

Furthermore, and referring to FIG. 22, the pump according to an exemplary embodiment is powered by a magnetic coupling. The pump includes a set of primary permanent magnets that are connected to a motor shaft of the motor drive. The pump further includes a set of secondary permanent magnets 300 that are embedded inside the pump impeller 270. The primary and secondary permanent magnets have poles that, when positioned in the pump, have opposite polarity facing each other. For example, the north polarity of the primary permanent magnet faces the south polarity of the secondary permanent magnet, or vice versa. Thus, the primary and secondary permanent magnets are electromagnetically attracted (i.e. magnetic flux). The motor drive rotates the primary permanent magnets, which causes the secondary permanent magnets 300 and impeller 270 to rotate.

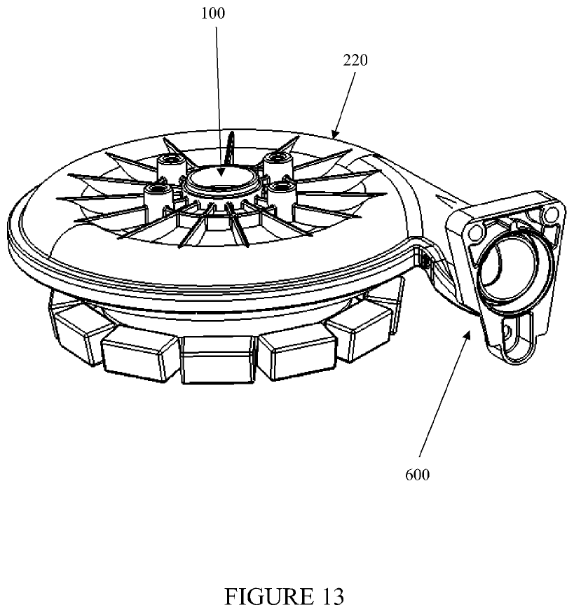

Now referring to FIG. 13, in certain configurations the pump may include an integrated electric motor-driven centrifugal pump. For example, the integrated electric motor-driven centrifugal pump includes permanent magnets 300 inside an impeller that act as a rotor of a motor drive. The motor drive has a housing in which an external wire winding is embedded. The external wire winding is a stator that provides an electromagnetic field to couple to the rotor to generate a torque to an impeller 270.

According to certain aspects of an embodiment of the blood pump device, the pump forms three flow paths formed by the pump chamber. In an exemplary configuration, blood enters from the inlet to the central hole of the pump impeller. Under the action of centrifugal forces, the blood is accelerated when moving radially along the impeller blade and reaches the maximum velocity to enter a peripheral volute 210 and then exit at the outlet. The primary flow path lies from the axial inlet to the tangential outlet through the impeller blade passage between the shroud 271 and impeller base 273. Furthermore, in certain configurations the primary flow path is formed by a top surface of the impeller (e.g., trailing edges of each blade) that are tangential at the exit to a center plane of the volute 230 of the housing 220 (peripheral volute). In an exemplary configuration, a secondary flow path exists in a gap 240/250 between the rotating impeller base 273 and the housing 220 to merge with the primary flow path in the central opening of the impeller. In certain configurations, the gap 240/250 is between approximately 0.5 mm and 2.0 mm. Further in certain configurations, a third flow path 260 is formed by the housing 220 and the shroud 271. For example, the third flow path 260 is generally formed by a flow domain between a top wall of the housing and a surface of the shroud.

Compared with the typical pumps, for example, the device according to aspects of the invention may have a shorter impeller base 273 and larger gap 240/250 between the housing wall 220 and the impeller base 273. The blade height of other typical pumps, as a further example, is smaller than that of the device configured in accordance with aspects of the invention that could contribute to higher shear wall stress (see FIG. 3a). A pump configured in accordance with aspects of the invention may have superior hemolytic and thrombotic biocompatibility because the flow pattern and shear level are optimal for the optimal geometry features including blade leading edge 272a height and angle, trailing edge 272b height and angle, volute 210 dimension, gap 240/250/260 sizes, etc. Thus, a pump configured according to aspects of the invention may provide superior hemolytic and thrombotic biocompatibility.

### Examples

Provided herein are non-limiting embodiments of the invention described above.

### Numerical and Experimental Methods

### Pump Descriptions

The Breethe pump (Breethe, Inc., Baltimore, MD) is a newly developed centrifugal pump featuring a single ball-and-cup hybrid magnetic/blood immersed bearing-supported impeller driven by a magnetically coupled motor drive (FIG. 1) formed in accordance with the foregoing description of the instant invention. A shrouded impeller with extended blades from the impeller hub and shroud is used. The primary flow path is from the inlet to the tangential outlet through the impeller blade passage between the hub and the shroud of the impeller. A U-shaped secondary flow path exists in the gap between the rotating impeller hub and the stationary pump housing and merges with the primary flow path through the central opening in the impeller. Permanent magnets are enclosed inside the impeller and coupled with the driving magnets which are fixed on an external pancake motor. The arrangement of the magnets enhances the stability of the rotary impeller and reduces the heat generated from the friction of the bearing. The Breethe pump weighs 49 g with a priming volume of 32 mL. The operational rotating speed of the Breethe pump is between 1000 and 5000 rpm, and the flow rate is up to 10 L/min. Both the CentriMag and Rotaflow pumps have a similar primary flow path from the axial inlet to the tangential outlet (FIGS. 2, 3) and a secondary flow in the gap between the rotating impeller and the pump housing. The impellers of the CentriMag and Rotaflow pumps also have a central opening for the secondary flow path to merge with the primary flow path. The CentriMag pump has an open impeller with extended blades while the Rotaflow pump has a shrouded impeller. The CentriMag pump weighs 67.3 g with a priming volume of 31 mL. It can provide high blood flow rate up to 9.9 L/min with a typical rotating speed up to 5500 rpm. The Rotaflow pump weighs 61.3 g with a priming volume of 32 mL. It can provide high blood flow rate up to 9.9 L/min with a typical rotating speed between 0 and 5000 rpm. More technical specifications and characteristics of the CentriMag and Rotaflow pumps have been described in detail elsewhere.

### Computational Fluid Dynamics (CFD) Analysis

The geometries of the three pumps were obtained from computer aided drawing (CAD) files or constructed by measuring the actual device components. Both structured and unstructured mesh were used in the flow domain. The details of the meshing procedure can be found in previous publications. Numerical simulations of flow inside the three pumps were conducted by using a commercial CFD package (Fluent 19.2, ANSYS, Inc, Canonsburg, PA). The flow field was obtained by numerically solving the flow fluid governing equations using the unstructured-mesh finite-volume-based commercial CFD solver FLUENT 19.2 (ANSYS Inc, Canonsburg, PA). Constant mass flow rate and 0 pressure boundary conditions were specified at the pump inlets and outlets, respectively. The walls of the three pumps were assumed to be rigid and no-slip. Blood was considered as an incompressible Newtonian fluid with the density of 1050 kg/m³ and viscosity of 0.0035 kg/m·s. The Semi-Implicit Method for Pressure Linked Equations (SIMPLE) pressure-velocity coupling scheme with second order accuracy was used to solve all fluid governing equations. The Menter's Shear Stress Transport (SST) k-ω model was used. Based on the suggested normal operating condition of the blood pumps, the volumetric flow rate of 5 L/min was prescribed as the inlet boundary condition and the pump pressure head was controlled at around 350 mmHg for numerical comparisons. The corresponding rotating speeds of the Breethe, CentriMag, and Rotaflow pumps were set as 3600, 4000, and 3600 rpm, respectively. The rotation of the pump impeller was modeled by using the sliding mesh approach. A mesh sensitivity analysis was conducted to ensure that the simulation results were independent of further mesh refinement. More details of the mesh sensitivity process can be found elsewhere. The final number of elements determined for Breethe, CentriMag, and Rotaflow pumps were 11.4, 7.3 and 9.4 million respectively. After the simulations converge, shear stress fields, residential time fields, and hemolysis indices can be calculated from the solved flow fields.

### Modeling of Shear Stress, Residence Time and Hemolysis

To assess the potential damaging effect of the NPSS inside the blood pumps, a viscous scalar shear stress was calculated based on the CFD solved flow fields. The residence time physically represents the length of time that blood has been in the pumps since it enters the inlet (in seconds) and it was calculated by using the Eulerian scalar transport equation. The pump with large residence time indicates bad washout. Hemolysis potentials of the pumps are estimated by using the hemolysis index (HI) (the percentage change in plasma-free hemoglobin (PFH) relative to the total hemoglobin).

### In Vitro Hemolysis Testing

A circulatory flow loop with ovine blood was constructed to evaluate the hemolytic performance of the three blood pumps. The tests were conducted following the protocol for assessment of hemolysis in continuous flow blood as suggested by the American Society of Testing and Materials (ASTM F1841-19). All the hemolysis tests were carried out with the flow rate of 5.0±0.2 L/min and the pump pressure head of 350±20 mmHg. The blood reservoir was immersed in a water bath to maintain the constant blood temperature of 37±1° C. The volumetric flow rates were measured by an ultrasonic flow probe (model 9PXL, Transonic Systems, Ithaca, NY) and the Transonic T410 flow meter (Transonic Systems, Ithaca, NY). The pump inlet and outlet pressures were measured by a calibrated piezoelectric pressure transducer (model 1502B01EZ5V20GPSI, PCB Piezotronics, Inc., Depew, NY).

Fresh ovine blood was collected from a local slaughterhouse. Heparin with the concentration of 10 U per 1 mL blood was added to prevent the blood from coagulation. The collected blood was filtered with a blood transfusion filter (PALL Biomedical, Fajardo, Puerto Rico) and Baytril solution (100 mg/mL, Bayer Corporation, Leverkusen, Germany) was added as an antibiotic. The filtered blood was then conditioned using phosphate buffered saline (PBS) (Quality Biological, Gaithersburg, MD, USA) to achieve a hematocrit level of 30±2%. The total plasma protein was adjusted to be above 5.0 g/dL. The blood pH level was maintained at 7.4±0.1 throughout the 6-hour experiment by adding bicarbonate solution.

Each mock circulation loop was filled with 0.5 L processed blood. Baseline (prior to the circulation) and hourly samples after circulation initiation were collected from the loop. The plasma of the collected blood samples was collected for the PFH measurement. The details of blood sample process and PFH measurement can be found in previous publications. The normalized index of hemolysis (NIH) was calculated based on the equation provided by ASTM F1841-19.

### Results

### Hydrodynamic Performance

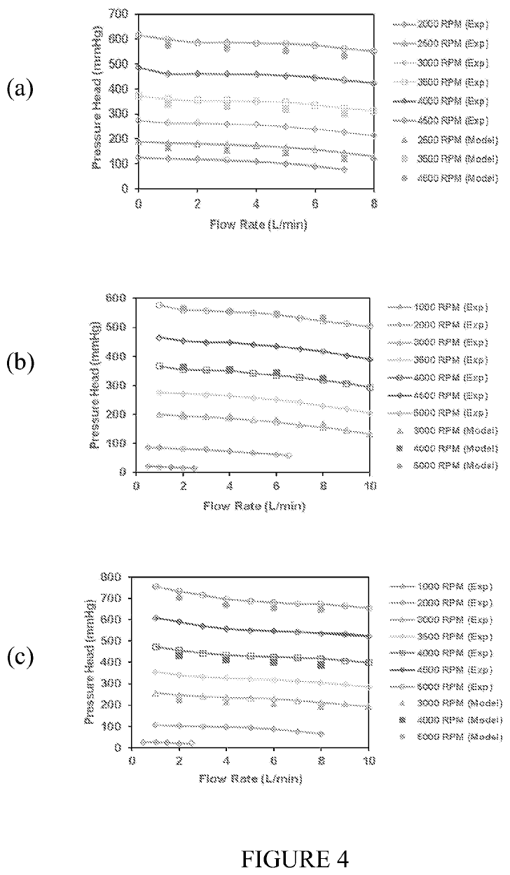

A set of rotating speeds and flow rates for the Breethe, CentriMag, and Rotaflow pumps were used for simulations. The CFD models were assessed by comparing the numerical prediction of pressure head of each pump with experimental measurement. The simulated and experimental measured pressure versus flow curves (HQ curves) of three pumps are shown in FIG. 4. The pressure heads (ΔP) generated by the three blood pumps under four flow rates at three rotational speeds were simulated. The numerically obtained ΔP values generated by the three blood pumps agree with the experimentally measured data under their operating flow rate and rotational speed. The relative error for each case is less than 10%. This indicated that the established CFD model can be used for further simulation.

### Flow Features

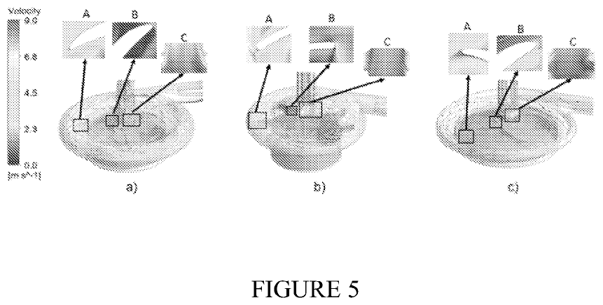

All the three centrifugal pumps have overall similar flow patterns, but different detailed features. Three flow paths exist in the pump chamber. Blood enters from the inlet to the central hole of the pump impeller. Under the action of centrifugal forces, the blood is accelerated when moving radially along the impeller blade and reaches the maximum velocity to enter the peripheral volute and then exit at the outlet. The primary flow path lies from the axial inlet to the tangential outlet through the impeller blade passage between the impeller shroud and impeller hub. As expected, a secondary flow path exists in the gap between the rotating impeller hub and the pump housing bottom and merges with the primary flow path in the central opening of the impeller. Another secondary flow path exists in the flow domain between the top housing wall and the shroud surface for the Breethe and Rotaflow pump or between the top housing wall and the axial tip of the impeller blades for the CentriMag pump. In all the three pumps a small area of flow separations were noted at the trailing edge tips of the impeller blades (FIG. 5a-5c, marker A). For the Breethe and CentriMag pumps, recirculation flows were observed at the leading edges of their impeller blades (FIG. 5a-5b, marker B).

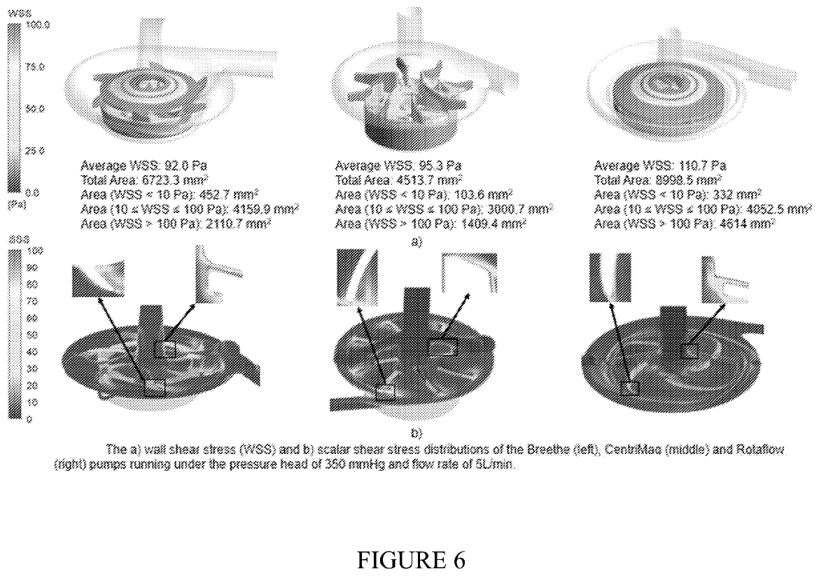

The wall shear stress (WSS) distributions on the impeller surfaces of the three blood pumps are shown in FIG. 6a. The WSS level was classified into three levels based on suggested impacts on blood cells and proteins, as follows: that 1) WSS<10 Pa, which is considered as the physiological shear stress (PSS); 2) 10 Pa≤WSS≤100 Pa, which may cause high-molecular-weight (HMW) VWF (von Willebrand factor) degeneration and platelet activation; 3) WSS>100 Pa, which represents the non-physiological shear stress (NPSS) that has been demonstrated to induce damage on blood components including blood cells and proteins. As shown in FIG. 6a, the NPSS (colored in red) are observed at the outer blade tip surfaces of the three pumps. For the Breethe and Rotaflow pumps with a shrouded impeller, NPSS are also observed on their shroud surfaces. Quantitatively, the Breethe pump had a relatively smaller area-averaged average WSS (92 Pa) compared with the CentriMag (95.3 Pa) and Rotaflow (110.7 Pa) pumps. More specifically, the Breethe, CentriMag and Rotaflow pump impellers had PSS distribution areas of 452.7 mm², 103.6 mm² and 332 mm², respectively. For WSS between 10 and 100 Pa, the WSS distribution areas of Breethe, CentriMag and Rotaflow pump impellers were 4159.9 mm², 3000.7 mm² and 4052.5 mm², respectively. As for the NPSS distribution areas, the corresponding values of the three pumps were 2110.7 mm², 1409.4 mm² and 4614 mm².

### Shear Stress Field and Residence Time

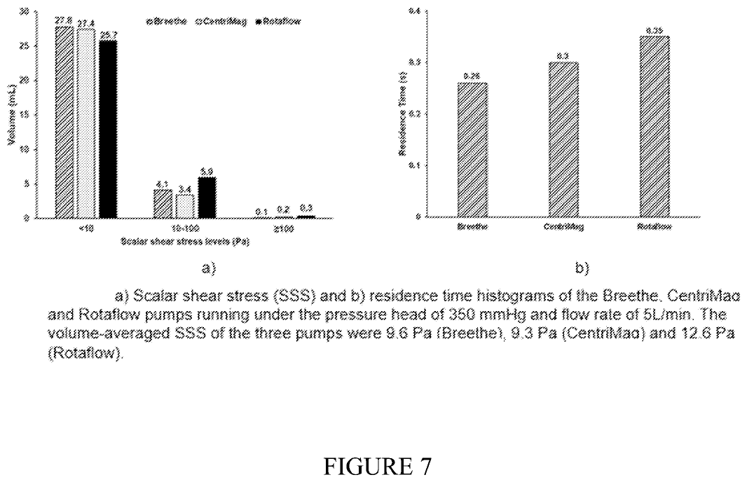

The scalar shear stress (SSS) distributions on a vertical midplane and a horizontal plane across the impeller blades of the three pumps are presented in FIG. 6b. High SSS appeared either at the trailing edge of the impeller blades or the primary and secondary narrow flow channels. The volumes of the blood exposed to different levels of SSS are shown in FIG. 7a. The majority of the blood volumes in all three pumps experienced the SSS less than 10 Pa. The Breethe pump has the smallest volume of 0.1 mL for SSS greater than 100 Pa (NPSS) while the CentriMag pump has the lowest volume of 3.4 mL for SSS between 10 and 100 Pa. The volume-averaged SSS for the Breethe, CentriMag and Rotaflow pumps were 9.6 Pa, 9.3 Pa and 12.6 Pa, respectively.

The velocity-weighted area-averaged residence times defined as the difference between the flow residential times measured at the outlets and inlets of the Breethe, CentriMag, and Rotaflow pumps under the tested operating condition (pressure head of 350 mmHg and flow rate of 5 L/min) were 0.26, 0.3, and 0.35 s, respectively. The residence times of the three pumps are given in FIG. 7b in which considering the three pumps had almost the same priming volumes, the Breethe pump might have better washout among the three pumps while the difference is not significant.

### Hemolysis Analysis

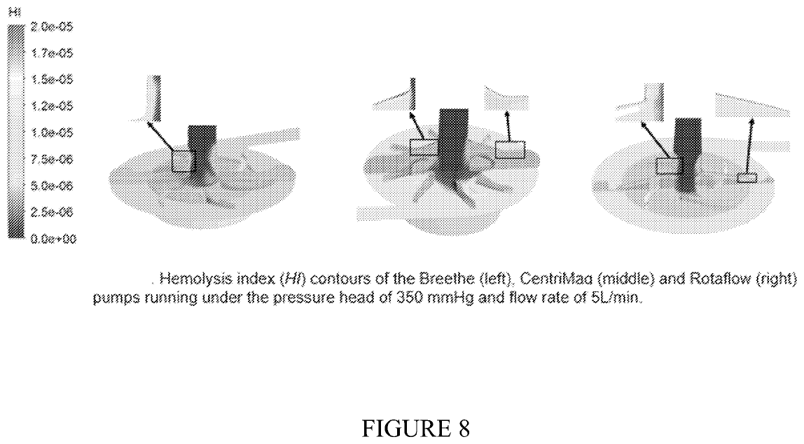

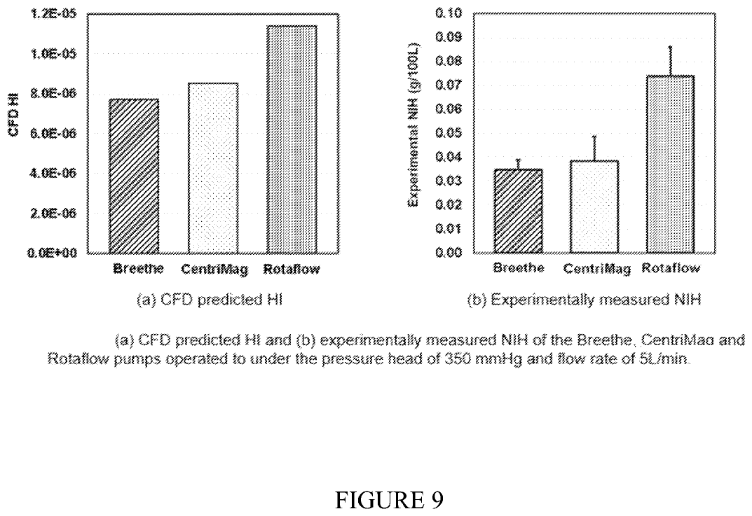

The calculated hemolysis index (HI) distributions on the mid and meridian planes of the three blood pumps are presented in FIG. 8. It was observed that there are high HI existing at the inlet or upper housing surfaces of the three pumps due to the high SSS in these regions (FIG. 6b). Overall, the Breethe pump generates relatively lower HI compared with the CentriMag and Rotaflow pumps. The HI levels at the outlet of the three pumps are shown in FIG. 9a. The Breethe pump generates a relatively low hemolysis index compared with those generated by the CentriMag and Rotaflow pumps (7.73×10⁻⁶ vs. 8.55×10⁻⁶ and 1.14×10⁻⁵). These computationally predicted HI levels are consistent with the experimentally measured NIH values for the three pumps as shown in FIG. 9b. The NIH value generated by the Breethe pump was the lowest among the three pumps when compared with the CentriMag and Rotaflow pumps (0.0347±0.0041 g/100 L vs. 0.0385±0.0101 g/100 L and 0.0739±0.0041 g/100 L).

### DISCUSSION

The flow dynamics of the new developed centrifugal Breethe pump operated under a clinically relevant operating condition for ECMO support or CPB (pressure head of 350 mmHg and flow rate of 5 L/min) was computationally analyzed with two clinically used pumps (CentriMag and Rotaflow). The flow features (velocity field, wall and scalar shear stress distributions) and device-induced hemolysis within the three pumps were assessed. The computationally predicted area-averaged WSS of the Breethe pump was relatively smaller than those of the CentriMag and Rotaflow under the same operating condition. This could be attributed to the unique impeller design (FIG. 1) of the Breethe pump. Compared with the CentriMag, the Breethe pump has a shorter impeller hub and larger gap between the housing wall and the impeller hub. The blade height of the Rotaflow pump is smaller than that of the Breethe pump, resulting in a narrow gap between its shroud inner surface and the top surface of the impeller hub, which could result in higher wall shear stress (FIG. 3a). The computationally predicted scalar shear stress distributions indicated that the overall bulk SSS in the Breethe pump was almost the same as the CentriMag pump and was still lower than the Rotaflow pump. Consistent with the shear stress assessment, the level of HI produced by the Breethe pump was lower compared with the CentriMag and Rotaflow pumps. The numerically calculated HI is dependent on both exposure time and SSS. The three pumps have almost similar priming volume and exposure time and were evaluated under the same operating condition. It is therefore anticipated that the structural design of the Breethe pump with lower WSS and overall SSS should help lower the hemolysis level. This computational prediction was confirmed by the experimentally measured NIH values for three pumps.

Although the computationally predicted HI using the CFD approach was not directly converted to corresponding experimental values for CFD model validation since the previous study showed that both the Eulerian scalar transport and Lagrangian models failed to reproduce the experimental results, it is still useful to use those methods to give relative comparisons of hemolysis in different blood pumps and combine the numerical results with experimental ones to assess and rank devices. The experimental and computational data about the CentriMag and Rotaflow pumps have also been recorded by other researchers. For example, Sobieski M A, Giridharan G A, Ising M, Koenig S C, Slaughter M S, Blood trauma testing of CentriMag and RotaFlow centrifugal flow devices: a pilot study, Artif Organs. 2012; 36(8): 677-82, also conducted hemolysis tests but their experimental results showed that the Rotaflow pump had a lower NIH compared to the CentriMag pump. This contradicting result could be attributed to the facts that: 1) they conducted their tests only twice (n=2) for each pump and the results were of less statistical significance when compared to the present study (n>6); 2) they used bovine blood instead of ovine blood in their case; 3) the operational conditions of the two pumps in their study were different (CentriMag: 3425 rpm, 4.2 L/min; Rotaflow: 3000 rpm, 4.17 L/min); 4) there was a lack of simulation results in their study to show the blood features of the two pumps and thus support the experimental data.

Having now fully set forth the preferred embodiments and certain modifications of the concept underlying the present invention, various other embodiments as well as certain variations and modifications of the embodiments herein shown and described will obviously occur to those skilled in the art upon becoming familiar with said underlying concept. Thus, it should be understood, therefore, that the invention may be practiced otherwise than as specifically set forth herein.

What is claimed is:

1. A blood pump device comprising:
a housing comprising a blood inlet and a blood outlet and defining a fluid pathway therebetween; and
an impeller within the housing, the impeller comprising a base, a base lid, two blades, and a shroud.

2. The blood pump device of claim 1, wherein the fluid pathway comprises a volute.

3. The blood pump device of claim 2, wherein the volute has a radius in any amount from 2 to 6 mm.

4. The blood pump device of claim 1, wherein an upper surface of the shroud extends downward at any angle from 0° to 30°.

5. The blood pump device of claim 1, wherein a lower surface of the base extends downward at any angle from 5° to 15°.

6. The blood pump device of claim 1, wherein an upper surface of the shroud and an interior surface of the housing form an upper gap.

7. The blood pump device of claim 6, wherein the upper gap has a width of any amount from 0.75 to 2 mm.

8. The blood pump device of claim 1, wherein a lower surface of the base and an interior surface of the housing form a lower gap.

9. The blood pump device of claim 8, wherein the lower gap has a width of any amount from 0.5 to 2 mm.

10. The blood pump device of claim 1, wherein a lower surface of the shroud and an upper surface of the base lid form an interior gap.

11. The blood pump device of claim 10, wherein the interior gap has a width in any amount from 1.5 to 6 mm.

12. The blood pump device of claim 1, wherein each blade has a leading edge and a trailing edge.

13. The blood pump device of claim 12, wherein each leading edge has a height in any amount from 3 to 9 mm.

14. The blood pump device of claim 12, wherein each trailing edge has a height in any amount from 1 to 5 mm.

15. The blood pump device of claim 1 further comprising a bearing, said bearing comprising a cup and a ball.

16. A blood pump device of claim 15, wherein the bearing is positioned between an interior wall of the housing and the impeller.

17. A blood pump system comprising:
The blood pump device of claim 1;
a motor positioned underneath the housing; and
a controller in communication with the motor.

18. The system of claim 17, further comprising magnets within a cavity formed by the base and the base lid.

19. A method of pumping blood, comprising the steps of:
providing the blood pump system of claim 17;
receiving at said controller a blood flow rate of blood flowing through the outlet;
receiving at said controller a rotational speed of the impeller; and
causing the controller to modify the rotational speed in response to the blood flow rate.

20. The method of 19, wherein the blood pump device further comprises a bearing, said bearing comprising a cup and a ball.
